(19) United States
(12) Patent Application Publication
(10) Pub. No.: US 2020/0245693 A1
(43) Pub. Date: Aug. 6, 2020

(54) ELECTRONIC CIGARETTE HAVING CONSTANT CONTACT SWITCH FOR VAPE “MOD” DEVICE

(71) Applicant: FAST EDDIE'S VAPE SHOP AND LOUNGE, LLC, Orlando, FL (US)

(72) Inventor: Matthew R. KLEIZO, Longwood, FL (US)

(21) Appl. No.: 16/263,000

(22) Filed: Jan. 31, 2019

(51) Int. Cl. A24F 47/00; A61M 11/04

(52) U.S. Cl. CPC A24F 47/008; A61M 15/06; A61M 11/042

(57) ABSTRACT

A personal vaporizer mod device includes a switch having a switch button member that carries a first magnet of first polarity. A switch insulator is secured within a switch body and has a central orifice and an internal counterbore section, which receives a switch firing pin and a second magnet having a polarity opposite the first magnet. The switch button member is repelled such that no contact exists between the switch firing pin and switch button member, but when depressed, the switch button member establishes electrical contact and current flows via a battery received within a mod housing to coils of the mod device.

### FIELD OF THE INVENTION

This invention relates to electronic cigarettes formed as personal vaporizers, and more particularly, this invention relates to a switch used in an electronic cigarette having a rebuildable atomizer, such as a rebuildable dripping atomizer (RDA).

### BACKGROUND OF THE INVENTION

Electronic cigarettes have become very popular in the last few years. Many of these electronic cigarettes operate as a battery-powered vaporizer that includes a mouth piece, a rebuildable device or deck, a juice or e-liquid that is vaporized and a heating element or similar atomizer, which is activated by a switch. The heating element is typically a heating coil and heats and vaporizes the juice. A wicking material helps draw the juice or liquid onto the coil such as from a well that holds the juice. In some devices, the user manually depresses a button on the side of the housing or at the end or bottom of the device to close the switch and actuate a contact button or “firing pin” or other contact device to complete the circuit between the battery and the atomizer.

The electronic cigarette designs currently in use vary and recent design modifications include mechanical “mods,” for example, that may have a rebuildable atomizer deck that allows a user to assemble or “build” the wick and coil themselves, instead of using off-the-shelf atomizer “heads.” These types of devices are often termed a rebuildable dripping atomizer (RDA), or sometimes referred to as a “dripper.” Experienced users of electronic cigarettes enjoy building their atomizers because they can choose a specific configuration and electrical resistance of their coil, and thus, pick their flavor and the amount of aerosolized vapor produced by the electronic cigarette. In the rebuildable dripping atomizer, the juice is usually dripped directly onto the coil and the wick. The liquid or juice used in these systems generally has vegetable glycerin or propylene glycol and may include flavoring. The RDA “building deck” or “deck” usually has two or three or more posts with holes formed in them that accept one or more coils. The user typically maintains the wick wet by dripping liquid onto the bare wick and coil and/or drawing juice from a well. Often a well-built RDA will last as many as 10 to 20 puffs and emit very large quantities of vapor.

When rebuilding the RDA decks, there is usually a positive post in the center and one or more negative posts located outside the center. One end or lead of the coil is attached to the positive post and the other lead of the coil is attached to one of the negative posts. This process is repeated on opposing sides if two coils are used. In some devices, air flow control can be maintained by spinning a top piece to close off the air holes or open them wide. The number of holes can vary so that the atomizer is used efficiently with one or two or more coils and the holes are lined up adjacent the coils. With a single coil, the RDA operates in a single coil mode, but with two coils, the RDA operates in a dual coil mode. Building the coils typically requires a user to select the type of wire and its gauge. In one example, the user wraps the wire around a cylindrical object to form the coil and then inserts the coil within the negative or positive leads in order to make a “build.” Often the device configuration does not permit an efficient electrical connection and makes the build difficult. Some designs are even clumsy to use after the build.

These “mod” devices may be side actuated or actuated from their lower end by depressing a button member or switch as part of a switch assembly to complete the electric circuit and actuate the coils for vaporizing the vape fluid. Often users desire a “feel” when depressing the button member that facilitates manipulation and vaping without requiring excessive force, but still allowing enough force to be applied to give a “feel” to the user where the button is not too easy to depress, which could create problems by actuating the coils by accident, and not too hard to depress, which makes actuation of the device difficult or bothersome. Also, some prior switch designs have been mechanically and/or electrically unreliable, some breaking or operating inefficiently, especially after repeated use. Other switch designs do not provide adequate user control over coil activation and vaping.

Therefore, there is a need for a switch assembly for a personal vaporizing mod device that meets the varied demands of users, gives a good “feel” during vaping, is mechanically reliable, and is less prone to damage and breakage after repeated usage.

### SUMMARY OF THE INVENTION

This summary is provided to introduce a selection of concepts that are further described below in the Detailed Description. This summary is not intended to identify key or essential features of the claimed subject matter, nor is it intended to be used as an aid in limiting the scope of the claimed subject matter.

A personal vaporizer mod device includes a mod housing that is electrically conductive in an example and comprises a closed upper end having a medially located orifice configured to receive the atomizer pin of a rebuildable or other atomizer deck that is supported on the upper end. An open lower end is configured to receive a battery having a battery contact that engages the atomizer pin for supplying electric current to the rebuildable or other atomizer deck for atomizing a vaping liquid.

A switch assembly is received in the open lower end of the mod housing which when actuated, completes an electrical circuit from the battery through the electrically conductive housing into the rebuildable or other atomizer deck. The switch assembly may comprise a switch body having first and second ends and an orifice defining respective first and second openings. The first end is received within the lower end of the mod housing. An internal shoulder stop is within the central orifice adjacent the second end. A switch button member is received within the orifice at the second end of the switch body and movable therein. It includes an outer shoulder that engages the internal shoulder stop and prevents removal of the button member from the second end. An annular well may be opposite the second end and form a central post. A first magnet of first polarity is received within the annular well.

A switch insulator is secured within the first opening of the switch body and has a central orifice and an internal counterbore section opposite the first opening. A shoulder is formed within the central orifice adjacent the internal counterbore section. A switch firing pin is received within the central orifice, and includes a notch that receives the shoulder and retains the switch firing pin within the central orifice. At least one second magnet is received within the internal counterbore section and has a polarity opposite the first magnet. The switch button member and its central post are repelled away from the switch firing pin. Upon depression of the switch button member, however, contact is made between the central post and switch firing pin to complete an electrical circuit contact with a battery received within the mod housing to energize the coils for vaping.

The mod housing may comprise a female tube member and male tube member forming a hybrid sleeve. The notch of the switch firing pin may be configured to engage the shoulder stop and limit upward movement of the switch firing pin out of the central orifice. The shoulder stop may include an upper shoulder ridge and the switch firing pin may include a firing pin head having a lower annular ridge that engages the upper shoulder ridge. The switch insulator may include an outer planar surface, and the switch firing pin includes a firing pin head extending above the outer planar surface.

The first magnet may be annular configured and may have an opening through which the central post of the switch button member passes. The at least one second magnet may be annular configured and have an opening through which the firing pin is received. The at least one second magnet may comprise two stacked magnets retained within the internal counterbore section. The switch button member may include a planar configured surface that a user depresses.

In yet another example, the switch assembly may be operative as a constant contact switch for the personal vaporizer mod device and include a switch body having first and second ends and an orifice defining respective first and second openings. The first end may include a first outer body section configured to be received within a mod housing, and an internal shoulder stop within the orifice adjacent the second end. A switch button member is received within the orifice at the second end of the switch body and movable therein. An outer shoulder engages the internal shoulder stop and prevents removal of the switch button member from the second end. An annular well is opposite the second end and forms a central post. A first magnet of first polarity is received within the annular well. A switch insulator is secured within the first opening of the switch body and has a central orifice and an internal counterbore section opposite the first opening. A shoulder is within the central orifice adjacent the internal counterbore section. A switch firing pin is received within the central orifice and includes a notch that receives the shoulder and retains the switch firing pin within the central orifice. At least one second magnet is received within the internal counterbore section and has a polarity opposite the first magnet, wherein the switch button member and central post are repelled away from the switch firing pin. Upon depression of the switch button member, contact is made between the central post and switch firing pin to complete electrical circuit contact with a battery received within the mod housing.

The outer planar surface of the switch insulator may be configured to receive a tool configured to remove the switch insulator from the switch body such as by unscrewing the switch insulator. The switch button member and switch firing pin may be formed of a conductive material. The switch body, switch button member and switch insulator may be substantially cylindrically configured. The switch button member may include a planar configured surface that a user depresses.

### DESCRIPTION OF THE DRAWINGS

Other objects, features and advantages of the present invention will become apparent from the detailed description of the invention, which follows when considered in light of the accompanying drawings in which:

FIG. 1 is an isometric view of the electronic cigarette showing an example RDA subassembly on the housing and the button or switch assembly as a constant contact switch in accordance with a non-limiting example.

FIG. 2 is an exploded isometric view of the electronic cigarette of FIG. 1 showing an RDA subassembly, the male and female tubes forming the housing, and the switch assembly as the constant contact switch.

FIG. 3 is a front sectional view of the electronic cigarette of FIG. 1 showing in greater detail the RDA subassembly, male and female tubes, battery and schematic representation of components in the switch assembly as the constant contact switch.

FIG. 4 is an exploded isometric view of the RDA subassembly of FIG. 1.

FIG. 5 is an exploded front elevation view of male and female tube members and having a unique configuration of orifices and ornamental design on the female tube member.

FIG. 6 is an isometric end view of the switch assembly as the constant contact switch showing the end of the firing pin.

FIG. 7 is an exploded isometric view of major components of the switch assembly of FIG. 6 as the constant contact switch.

FIG. 8 as a sectional exploded isometric view of the switch assembly shown in FIG. 7.

FIG. 9 is a front elevation view of the switch assembly as the constant contact switch in accordance with a non-limiting example.

FIG. 10 is a sectional view of the switch assembly taken a long line 10-10 of FIG. 9.

FIG. 11 is an isometric sectional view of the switch assembly of FIGS. 9 and 10.

FIG. 12 is another sectional view of the switch assembly similar to that shown in FIG. 10 and showing the constant contact switch in an open position.

FIG. 13 is another sectional view of the switch assembly similar to that shown in FIG. 12, but showing the constant contact switch in a closed position.

FIG. 14 is an enlarged schematic view of the switch firing pin, switch insulator and magnet showing the relative spacing of the components.

### DETAILED DESCRIPTION

The present invention will now be described more fully hereinafter with reference to the accompanying drawings, in which preferred embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Like numbers refer to like elements throughout.

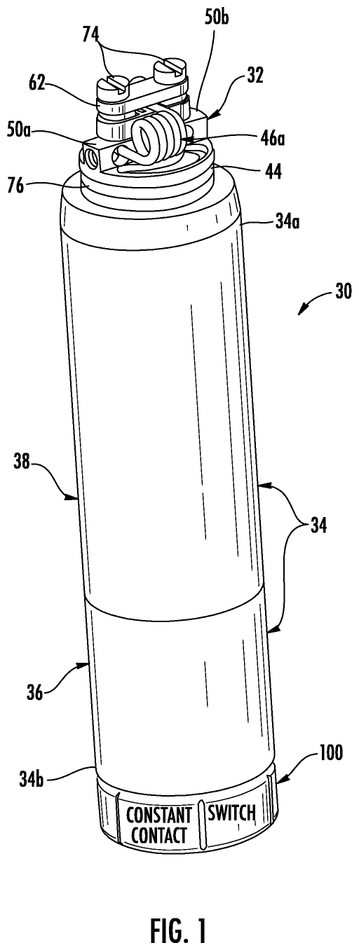

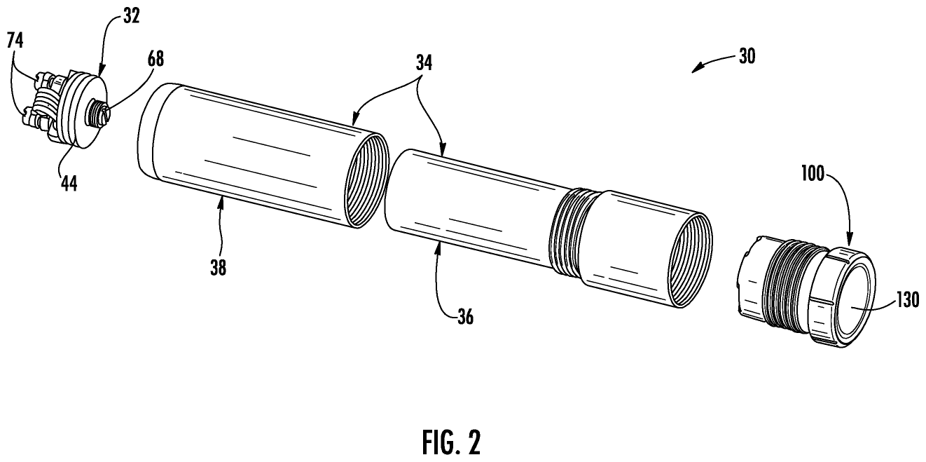

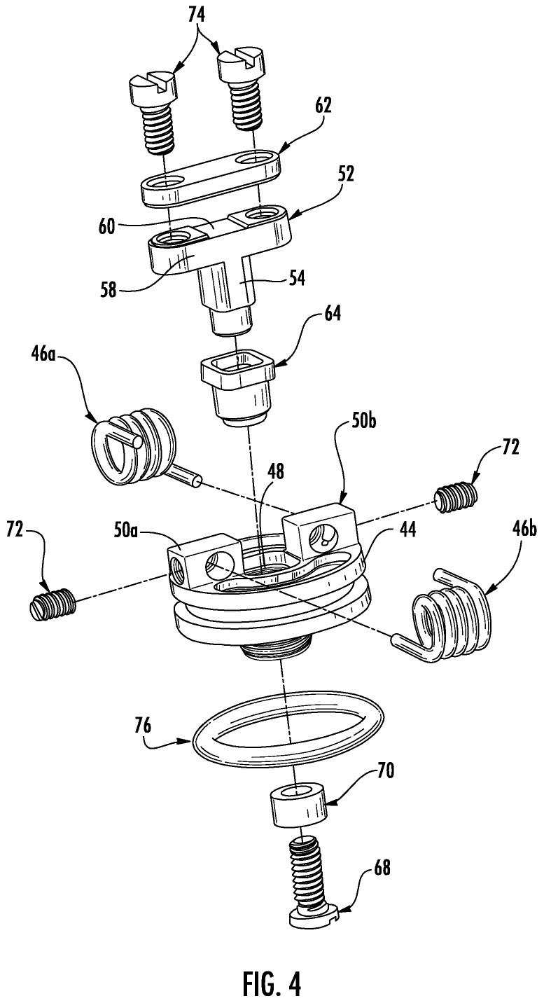

Referring now to FIGS. 1-4, there is illustrated the assembled electronic cigarette as a “mod” device generally at 30 that includes three major subassemblies as the rebuildable dripping atomizer (RDA) subassembly shown generally at 32, and in exploded isometric at FIG. 4, a housing subassembly referred to also as the “mod” housing and generally indicated at 34 and which can be formed as a monolithic, one-piece structure or formed from male and female tubes 36,38 as illustrated (FIGS. 2, 3 and 5), and a switch assembly as a constant contact switch, also referred to as a button subassembly indicated generally at 100, and operative as a switch for completing the electrical circuit for vaping such as by depressing a switch button member as explained below. The switch assembly as the constant contact switch 100 operates as a switch for vaping and completes the electrical circuit for operating the electronic cigarette and its functionality is explained in further detail below.

The button or switch assembly is shown generally at 100 and improves operator “feel” and is mechanically durable. It operates as a constant contact switch, requiring constant user operation, i.e., switch depression for coil energization from the resulting electrical circuit completion with a battery. Further details of the switch assembly 100 are explained below after an explanation of other components of the device 30.

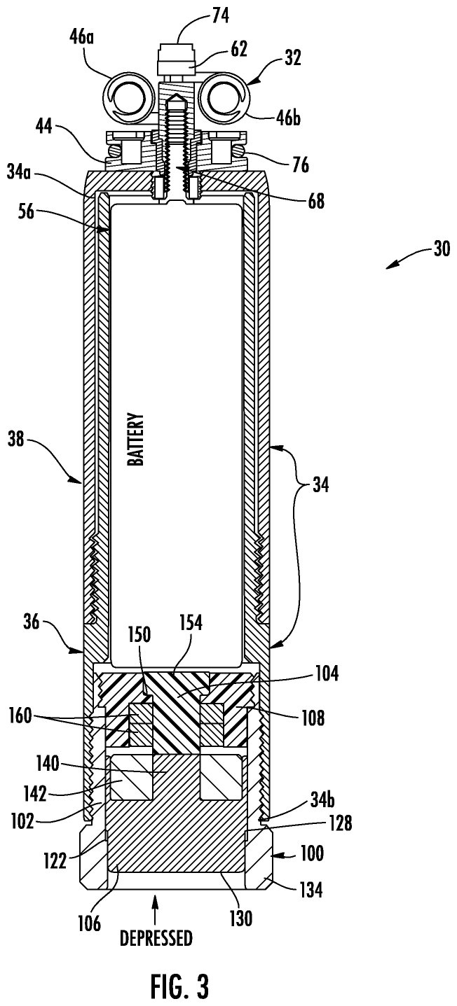

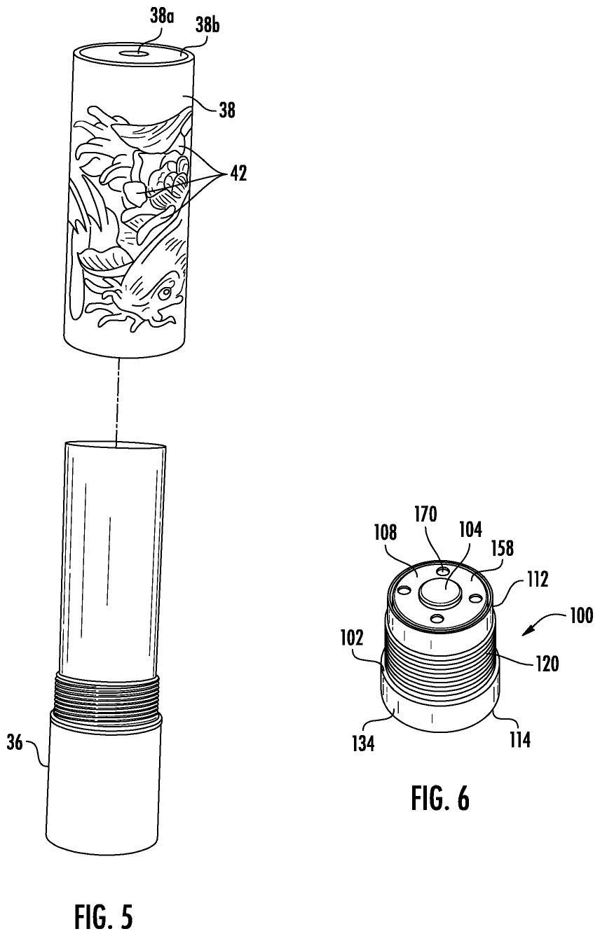

As shown in FIGS. 1-3, the mod device 30 includes a mod housing 34 that is cylindrically configured as a tube, which in this example includes the male and female tubes 36,38, and formed of a conductive material such as brass, stainless steel, copper, or other metallic material, and has first and second ends 34a, 34b and configured to hold a battery 56 (FIG. 3) having a positive pole adjacent the first end of the housing near the RDA subassembly 32 and a negative pole adjacent the second end of the housing where the switch assembly as the constant contact switch 100 is located. In this embodiment, the housing 34 includes the male and female tubes 36,38 and in a unique design as an example, the female tube 38 has through-holes or orifices 42 (FIG. 5) configured to expose portions of the male tube 36 so that ornamental effects located on the male tube can be seen through the orifices of the female tube. Also, the configuration of the holes or orifices 42 may add to the ornamental appearance and have other functionality such as venting of any battery gas that may develop. Thus, a wide variety of ornamental features in the form of orifices or through-holes 42 (openings) may be added to the body of the female tube 38 to expose ornamental features on the male tube 36. The combination of features between the male and female tubes creates a secondary layer that is otherwise obscured. Additional ornamental features or colors may be added to provide a higher level of decorative aesthetic features.

As illustrated, the switch assembly as the constant contact switch 100 includes a threaded portion (FIGS. 2, 3 and 6) as part of the switch body 102 that is received within a threaded portion of the male tube member 36 and carries via other components explained below the contact button or firing pin 104 or other contact mechanism that is actuated when a switch button member 106 is depressed to complete the circuit in this example. This switch assembly as the constant contact switch 100 in this example includes this solid button piece as the switch button member 106 that is pushed manually by depressing with a finger, such as the pinky, and thus, moving components of the switch button member into the firing pin 104, and thus, very slightly into the terminal battery 56 (FIG. 3), thus, energizing heating coils for vaping.

Referring to FIGS. 1-4 and especially the exploded isometric in FIG. 4 of the RDA subassembly 32, this subassembly includes a cylindrically configured RDA deck 44 formed from a conductive material such as stainless steel, brass, copper, or other metallic material, and supported at the first end 34a of the mod housing 34 and configured to support first and second heating coils 46a, 46b, each being a single large filament as a heating coil or a number of individual filaments wound together. The RDA deck 44 includes a central orifice 48 and first and second spaced terminal posts 50a, 50b configured to connect to first ends of respective first and second heating coils 46a, 46b using a mechanical attachment mechanism.

Further details of the RDA subassembly 32 are described in commonly assigned and co-pending U.S. patent application Ser. Nos. 15/921,926 filed Mar. 15, 2018 (U.S. Patent Publication No. 2018/0263285); and 16/029,730 filed July 9, 2018 (U.S. Patent Publication No. 2018/0310630); the disclosures which are hereby incorporated by reference in their entirety. Further explanation and general details of the RDA subassembly 32 are given below to explain its function as the “mod” device 30, but reference should be made to the incorporated by reference '926 and '730 applications for further details. It should be understood that many different RDA subassemblies of different configurations and coil designs may be connected to the housing 34, which incorporates the switch assembly 100. The illustrated RDA subassembly 32 is only one example.

A central column terminal 52, also referred to as a T-column, includes a central post 54 received within the central orifice of the RDA deck 44 that engages the positive pole of a battery 56 held within the housing 34 and a transverse support 58 formed as an upper “T” of the “T-column” carried by the central post 54 and spaced above the terminal posts 50a, 50b and on which second ends of respective first and second heating coils 46a, 46b are positioned such as best shown in FIG. 4, where the ends may be positioned on a compression slot formed thereon.

A clamp bar 62 is secured onto the transverse support 58 and fixes the second ends of the heating coils 46a, 46b on the transverse support 58. An insulator 64 in one example is formed as a sleeve (FIGS. 3 and 4) and is received within the central orifice 48 of the RDA deck 44 and receives the central post 54 and separates and electrically insulates the central post 54 from the RDA deck 44. It is formed from a plastic or other insulator material. As shown in FIGS. 3 and 4, the RDA deck 44 includes a cylindrically configured and recessed well 66 below the terminal posts 50a, 50b to hold the juice used for vaping, for example. The terminal posts 50a, 50b are formed on the upper circumferential edge of the RDA deck (FIG. 4) so that the well 66 has a large annular configuration that is deep, and in an example, has an internal stepped configuration.

The switch assembly as the constant contact switch 100 operates as a switch and is located at the second end 34b of the housing, and when closed, the switch completes an electrical circuit from the negative pole of the battery 56 at the second end 34b of the housing and through the housing 34 to the RDA deck 44 and energizes the heating coils 46a, 46b to vaporize the juice contained on at least one wick, and in the illustrated example, the wicks held by the heating coils. In this illustrated example of FIGS. 3 and 4, the central post 54 includes a contact screw 68 that engages the battery 56, such as the positive terminal at the top of the battery, and is adjustable relative to the battery to ensure electrical contact when the switch is closed. A sleeve ring 70 formed from an insulator material such as plastic encompasses the contact screw 68 to separate and electrically insulate the contact screw from the RDA deck 44. Set screws 72 (FIG. 4) may secure the first ends of the heating coils 46a, 46b onto the respective terminal posts 50a, b and clamp screws 74 may secure the clamp bar 62 to the transverse support 58.

The RDA deck 44 includes an annular outer surface and supports at least one O-ring 76 on the outer surface to retain an RDA cap and a drip tip in an example (not shown) that are received over the RDA deck. The transverse support 58 includes the compression slot 60, as best shown in FIG. 4, into which the second ends of the heating coils 46a, 46b are received to aid in securing the second ends of the respective first and second heating coils. The heating coils 46a, 46b are also sometimes referred to as wire filaments by some skilled in the art since the heating coils are small gauge wires. The heating coil may also be formed from a plurality of wire filaments that are wound together. Typically, a heating coil is one gauge and a small diameter of wire or filament that is formed of a material having properties that allow it to heat and vaporize the juice when a current flows through the wire.

The RDA deck 44 is formed from an electrically conductive material such as brass, stainless steel, copper, or other metallic material, and stores a prescribed amount of juice or e-juice as it is sometimes referred as the liquid in the recessed well 66. The RDA deck 44 operates as a base upon which other components of the RDA subassembly 32 attach through various mechanical fastening techniques such as the described set screws 72 and clamp screws 74 (FIG. 4). The lower portion of the RDA deck 44 includes a mechanical attachment to the female tube 38, the preferred embodiment of which is a male threaded fastener that mates with a female threaded orifice on the top portion or first end 34a of the female tube 38 of the housing 34 so that the RDA deck is secured to the housing via the threaded engagement, thereby creating an electrical connection between the RDA deck 44 and the female tube 38 and the overall housing. The RDA deck 44 includes the recessed well 66 as described before that is of a depth sufficient to retain a volume of fluid as the juice for “vaping.” The recessed well 66 in one example is annular configured within the RDA deck 44 and positioned below the terminal posts 50a, 50b and may be of any singular profile or stepped profile where the various shoulder portions forming the stepped profile are configured to any desired depth within the body member forming the RDA deck 44. This profile helps retain the juice and wicking of juice on the wicks. As also shown in the various drawings, the RDA deck 44 may include one or a plurality of recesses or grooves on the outer peripheral surface, such as the illustrated groove formed around the perimeter or outer surface, to receive and retain one or more O-rings 76 that help retain the RDA cap.

In another embodiment, two or more grooves or other receiving recesses in the RDA deck 44 may receive a plurality of O-rings and retain a RDA cap and sometimes drip tip (not shown) over the RDA deck 44 as known to those skilled in the art. The RDA deck 44 includes in this example the electrical terminal posts 50a, 50b formed above the recessed well 66, on an upper annular top surface or edge surface forming the RDA deck. The two opposing electrical terminal posts 50a, 50b are of a negative polarity and positioned in-line with the central column terminal 52 as the configured T-column and the clamp bar 62 to connect to first ends of respective first and second heating coils 46a, 46b, which could be formed in an example as a number of filaments or a preferred single gauge wire that has sufficient resistance for heating and vaporizing the juice when the electrical circuit is closed.

The first and second terminal posts 50a, 50b may each include a primary smooth bore as a through-hole through which a free end of one or more wire filaments or heating coils 46a, 46b may be inserted and passed through as illustrated in the drawings. A secondary blind hole as a preferred female threaded fastener blind hole intersects the through-hole that had received the heating coil, and in turn, receives a set screw 72. This blind hole may be oriented at an angle relative to the primary smooth bore as the through-hole that receives the heating coil such that a set screw 72 is screwed in as a mechanical attachment and creates a compressive force upon the free end of the wire filaments or heating coil against the respective terminal post 50a, 50b, thereby creating an electrical connection between the RDA deck 44 and the one or more wire filaments or heating coil. The RDA deck 44 includes the central orifice 48 as a through-hole of varying cross-sections through which the central post 54 of the central column terminal 52 as the T-column is received. It also receives the sleeve 64, the sleeve ring 70, and contact screw 68 that are assembled together. The central orifice 48 as a through-hole of the RDA deck 44 may include a plurality of stepped shoulders forming a stepped configuration upon which the compressive forces imposed by the mechanical arrangement of the sleeve 64, sleeve ring 70, central post 54 of the central column terminal 52 and contact screw 68 may be applied to aid in retaining those components together.

The at least one O-ring 76 may be formed from an easily compressible and elastic material and serve to create an air-tight seal between the RDA cap and drip tip (not shown) that are commonly used in the art and operate as an assembly technique between the RDA deck 44 and the common RDA cap via a compression fit interaction. The at least one O-ring 76 is received along the groove or other recess that extends along the outer peripheral surface of the RDA deck 44 and is held in place by the contours of the groove and the tensile elastic deformation of the O-ring.

The sleeve 64 is an insulator as described above is formed from an electrically insulating material and could be formed from one or more components. It serves as an electrical insulator to ensure no physical contact is made between the central column terminal 52 and the contact screw 68 and the RDA deck 44. In one example, it encompasses all or portions of the central post 54 and portions of the contact screw 68 that would otherwise make physical and electrical contact with the RDA deck. The bottom of the sleeve 64 on both the inside and on its orifice may include a shoulder or other stepped portion forming a profile to engage a stepped portion of the RDA deck central orifice 48 and a stepped portion of the central post. The sleeve 64 is inserted through the through-hole, i.e., central orifice 48 of the RDA deck from above. The sleeve 64 includes a through-hole of varying cross-sections through which the central post 54 and contact screw 68 are inserted and assembled via a mechanical technique. The sleeve 64 as noted before includes a plurality of shoulders or other stepped profile features both internally and externally upon which the compressive forces of the mechanical assembly of the RDA deck 44, sleeve ring 70, central column terminal 52, and contact screw 68 are applied.

The contact screw 68 (also termed by some as a 510 pin) is also made from an electrically conductive material and could be formed by one or more components along with the other conductive components, to transmit electrical energy from the battery terminal as the positive terminal in this case to the heating coils 46a, 46b. The contact screw 68 is inserted into the through-hole of the sleeve ring 70 and the combination of the two then inserted into the through-hole as the central orifice 48 of the RDA deck 44 from below and then further assembled by mechanical attachment to the central post 54 and sleeve 64. The contact screw 68 serves as one of the two electrical contacts for the battery 56 and maintains a constant electrical connection with the battery when the apparatus as the electronic cigarette is fully assembled.

The clamp screws 74 are also made from an electrically conductive material to affix the clamp bar 62 to the transverse support 58 as the top “T” of the central column terminal 52, also termed the T-column, via a mechanical attachment using the clamp screws 74 in a preferred embodiment, which simultaneously apply compressive forces between the clamp bar and the central column terminal such that force is applied to the free second ends of the heating coils 46a, 46b, thereby retaining and affixing free ends of the wire filament or heating coil in place. This creates an electrical connection between the central column terminal 52 and clamp bar 62 and heating coils 46a, 46b. The clamp bar 62 is one component in a series of components used to transmit electrical energy from the battery terminal to the wire filaments or heating coil.

The central column terminal 52 that is also referred to as the T-column is formed from a conductive material such as stainless steel, brass, copper, or other metallic or conductive material, and used as an intermediary component to transmit the electrical energy of the battery to the heating coils 46a, 46b while insulated against electrical contact with the RDA deck 44 via the sleeve 64.

The heating coils 46a, 46b may be formed from multiple strands of wire filaments or one wire as a single gauge forming a heating coil as a single strand, and of course, from an electrically conductive material that has the properties and resistance to heat up when the switch is closed and atomize fluid on a wick. The physical connection is made at either free end to create an electrical connection where the electrical energy is passed through the heating coils upon activation of the device as an electronic cigarette by the user. This can occur in one example by depressing their pinky finger against the lower end of the constant contact switch 100 as a switch assembly and on the switch button member 106 (button) that in an example is positioned at the second end of the housing and explained above. The natural electrical resistance of the wire filament as a heating coil, in an example, generates heat sufficient to vaporize a prescribed liquid substance such as the juice contained on a wick, in one example, and contained in the recessed well 66. Different flavors of juice may be used and different types of juice. The heating coils 46a, 46b have one free end affixed to the RDA deck 44 via its electrical terminal posts 50a, 50b and the mechanical attachment via the set screws 72 (FIG. 4). The opposing free end is affixed to the transverse support 58 of the central column terminal as the T-column and by the clamp bar via the assembled compressive forces.

The set screws 72 may be made of an electrically conductive material and aid in creating an electrical connection between the free end of the heating coils 46a, 46b and the electrical terminal posts of the RDA deck. This is accomplished by passing the free end of the heating coils 46a, 46b through or within the through-hole of the electrical terminal first and second posts 50a, 50b and using a mechanical attachment via the set screws into the electrical terminal post blind holes, which intersect the through-holes, to compress, retain and affix the free ends of the heating coils.

The battery 56 serves as a source of stored electrical energy to power the device as the electronic cigarette as the “mod” device 30. The battery 56 can be any form that can be held within the cylindrical housing 34 formed from the female tube 38 and male tube 36.

The male-female tube subassembly forming the mod housing 34 includes the female tube 38, which is preferably made from an electrically conductive material as noted before and uses a housing or storage as the male tube for the battery 56 as shown in FIG. 3 and as an intermediate component to transfer the electrical energy of the battery from one terminal of the battery to the switch assembly 100 as the constant contact switch forming a bottom housing button subassembly and through the male and female tubes 36, 38 to the RDA subassembly 32 where it ultimately flows back to the opposing terminal of the battery. The female tube 38 uses mechanical attachment techniques to attach to the RDA deck 44 of the RDA subassembly 32, the preferred embodiment of which is a female threaded fastener. The female tube may have the plurality of through-holes 42 on any portion of its length of any size or shape that can be configured to give a unique design and permit the otherwise obscured portion of the male tube to be seen and give a further decorative ornamental effect by allowing the surface of the male tube to be exposed.

The male tube 36 is also made from an electrically conductive material and used as part of the mod housing for the battery 56 and as an intermediate component to transfer the electrical energy of the battery from one terminal of the battery to the constant contact switch as the switch assembly 100 and to the female and male tubes to the RDA subassembly 32 where the current flows back to the opposing terminal of the battery 56. The male tube 36 uses a mechanical attachment as threads in this example to attach the constant contact switch as the switch assembly 100 to its lower end, the preferred embodiment which is a female threaded fastener shown in FIGS. 2 and 3. The male tube 36 mechanically attaches to the female tube 38, the preferred embodiment of which is a male threaded fastener. The male tube 36 inserts within the volume of the female tube 38 as best shown in FIG. 3, which partially or wholly obscures a portion of the male tube's length, depending on the geometry of the female tube.

The switch assembly as the constant contact switch 100 operates as a switch sufficient to open and close an electrical circuit, acting as a normally open electrical switch between the positive terminal of the battery 56 and its opposing negative terminal as explained above. The switch assembly 100 mechanically attaches by threading into the male tube 36 and an electrical connection is created when the switch is closed that completes the electrical circuit from the battery 56 through the tube to the RDA. The user of the electronic cigarette as the “mod” device may depress using their finger, such as their pinky, the switch button member 106 (also referred to as “button” by some) and selectively open or close the electrical switch, thereby activating or deactivating the device 30 as the electronic cigarette and energizing or de-energizing the device.

One advantage of positioning the terminal posts 50a, 50b of the RDA assembly 32 in line with the central column terminal 52 and clamp bar 62 as shown in FIG. 4 is that it creates an arrangement where one free end of the heating coils 46a, 46b may be inserted into either of the electrical terminal posts, at the discretion of the user, and the opposing free end of the heating coils may be simultaneously inserted into the compression slot 60 formed on the top of the transverse support 58, thereby reducing the difficulty of assembly. Another advantage of this preferred embodiment's arrangement is that all of the electrical connection features are positioned above the recessed well 66, thereby maximizing the available volume of fluid storage within the recessed well.

An advantage of having the male tube 36 inserted within the female tube 38 is that a wide variety of ornamental features in the form of through-holes 42 can be added to the body of the female tube, which then will expose the male tube's otherwise obscured portions for ornamental or decorative effects. This combination of the features between the male and female tubes 36,38 creates a secondary layer for the otherwise obscured portions of the male tube, upon which ornamental features or colors may be added to provide a higher level of decorative aesthetic when compared to other devices.

In an example, the RDA deck 44 may be 22 mm and include 2.5 mm post holes to fit all popular gauges of wire. It can include titanium screws and has an easy building area. The terminal posts can be milled to the deck to cut down on spinning or breaking. The positive center has a keyed bottom to prevent spinning. The titanium screws add to an aesthetic appeal. A battery may be added to the housing by unscrewing the switch housing as the constant contact switch 100 and inserting a battery. An example could be a high drain 40 amp battery with the positive end in first.

Different types of heating coils “vape wires” may be used, including Kanthal (FeCrAl), Nichrome, stainless steel, nickel, and titanium. Stainless steel is considered by many to be the most versatile. Different grades of stainless steel wire may be used. The most popular gauges for the wire are 32, 30, 28, 26, 24, and 22. The type of wicking material can vary and could include organic cotton, Japanese cotton pads, ekowool, silica, and rayon fiber.

As noted in the incorporated by reference '926 and '730 patent applications, it is possible to employ a three-piece button assembly that could operate with the switch assembly 100 that includes a firing pin mechanism as described above.

The electronic cigarette as the “mod” device 30 as described above may also be described as a personal vaporizer that operates an inhaler for “vaping” and of course, for personal use. It uses the described RDA subassembly 32, which is connected by a threaded connection in a central threaded orifice 38a onto the female tube 38, also described as a female tube member, which operates as a sleeve to receive the male tube 36, also referred to as the male tube member. The term “member” can be used with and without the term male tube or female tube. The male and female tube members 36,38 are typically formed of a metallic material, but could be metal plated plastic in an embodiment or other materials as long as some conductive path can be established with the battery 56, switch assembly 100 and RDA subassembly 32. The metallic material could be copper, brass, aluminum, or other materials. The female tube member 38 can be referred to as a hybrid sleeve, since the female portion will accept the RDA subassembly 32, such as via a commonly referred to 510 connection, and thus, the top cap with the hybrid sleeve as the female tube member is combined with the male tube member, which is received within the female tube member operating similar to a sleeve. It should be understood that different rebuildable atomizers (RBA's) may be supported by and connected to the female tube member 38 and a preferred RDA is described above, rebuildable tank atomizers may be attached.

The housing 34 formed of the female tube member 38 and male tube member 36 and switch assembly 100 may also be referred to as a personal vaporizer “mod” device as noted before, while without the switch assembly 100, the housing as the male and female tube members could be referred to as the personal vaporizer mod device housing or mod housing. It should also be understood that the RDA subassembly 32 as described and the switch assembly 100 as the constant contact switch as described above and in greater detail below could be used with a one-piece housing.

The personal vaporizer 30 as an electronic cigarette described above relative to FIGS. 1-4 can be referred to as a hybrid mod since the RDA assembly 32 will thread via what is termed the conventional 510 connection via a centrally located threaded orifice 38a onto the substantially planar upper end as a top flat portion 38b of the female tube member 38, which is part of the housing 34 and includes the male tube member 36. The bottom portion of the RDA deck 44 seats on top of the flat or planar upper end 38b of the female tube as a “sleeve” or “hybrid sleeve,” which receives the male tube member 36.

This design is also considered and referred to as a “Purge®” mod. The male tube member 36 may have a lower portion with a distinct design and the upper section may have a distinct coloration. The decorative design on the lower portion of the male tube member 36 may be exposed once the male tube member is screwed into the female tube member 38 shown in FIG. 5, which includes a specific ornamental design configuration and a distinct set of orifices or openings 42 forming a specific design.

It is possible to accommodate at different sized batteries by a selected size battery adapter as a battery sleeve, such as for a 18650 battery. A battery adapter or sleeve may be received within the male tube member 36. Because the switch assembly as the constant contact switch 100 has its switch button member 106 threaded into the lower end of the male tube member 36, it is possible to form components where the top portion presses up against the battery (or battery adapter) such as by stand-offs and pushes the positive pole of the battery upward into the contact screw 68, also termed the 510 pin, and helps prevent battery “rattle.” As noted before, the personal vaporizer may be compatible with 18650 and 20700 and other batteries.

This female tube member 38 as a sleeve-top cap design or hybrid sleeve, as it is also termed, is an excellent design that allows the ornamental effects to be enhanced via deep colors on the upper portion of the male tube member 36 that are exposed via the orifices or openings 42 in the female tube member 38 for ornamental effects and designs such as shown in FIG. 5. These openings 42 may also function as vent holes. Artwork of different designs can be CNC machined deep into the tube.

As shown in FIG. 5, a different mod housing 34 is illustrated with the female tube member 38 disassembled from the male tube member 36 (FIG. 5) and showing the top surface of the switch assembly 100 (FIG. 6) and its switch firing pin 104 located in the center of a switch insulator 108 that is described below. The female tube member 38 in this example has a specific ornamental configuration for its openings 42 and a different surface configuration and design. A swirl or other design may extend along the lower section that is exposed on the male tube member 36 and on the lower exposed section of the switch assembly as the constant contact switch 100. Vent holes 42 may be formed in the housing.

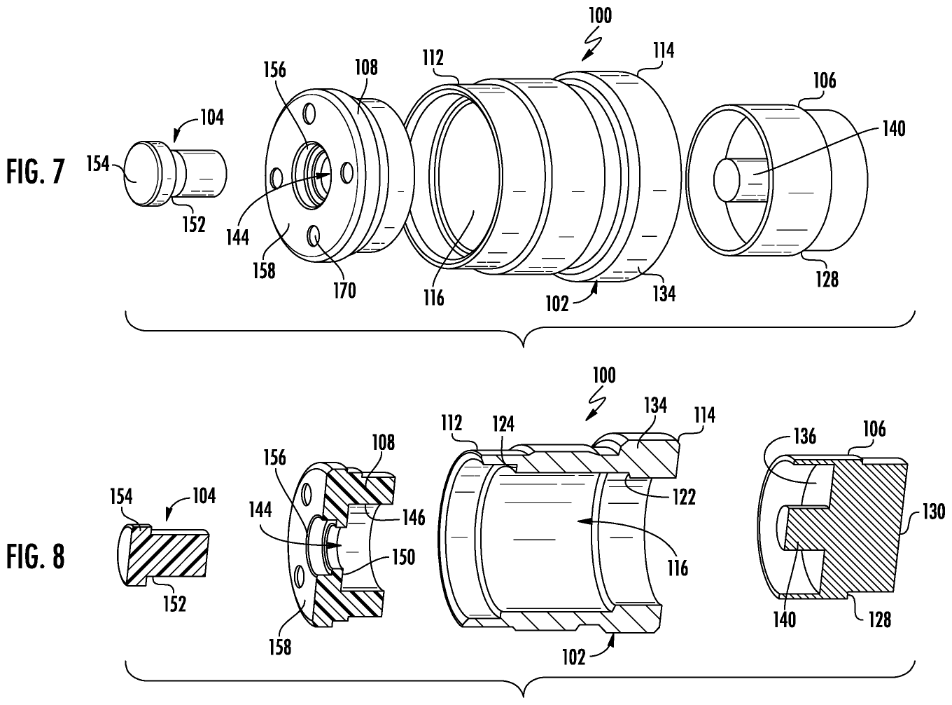

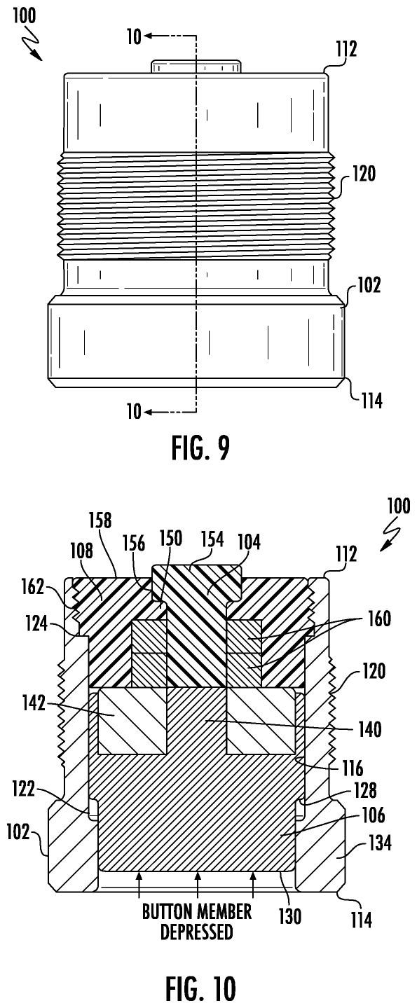

Referring now to FIGS. 7 and 8, there are illustrated the major components of the switch assembly 100 operative as a constant contact switch for the personal vaporizer mod device 30 such as shown in FIG. 1. The primary components include the switch body 102, switch button member 106, the switch insulator 108 and the switch firing pin 104. These components interoperate with a first magnet 142 of first polarity carried by the switch button member 106 and at least one second magnet 160 having a polarity opposite the first polarity and carried by the switch insulator 108 as will be explained in greater detail below and shown in FIGS. 10-14.

The switch body 102 is substantially cylindrically configured as illustrated and has first and second ends 112,114, also referred to as upper and lower ends, and an orifice 116 defining respective first and second openings. The first end 112 includes a first outer body section that is configured to be received within a mod housing, and as shown in FIG. 10, and includes threads 120 to allow the switch body 102 to be screwed into the lower end of the mod housing, such as the male tube 36 as shown in FIGS. 1-3. The switch body 102 includes a first internal shoulder stop 122 within the orifice 116 adjacent to the second end 114 as shown in FIGS. 8 and 10-13. A second internal shoulder stop 124 is within the orifice 116 adjacent the first end 112 as also illustrated. The switch button member 106 is received within the orifice 116 at the second end of the switch body 114 and moveable therein. The switch button member 106 includes an outer shoulder 128 that engages the first internal shoulder stop 122 adjacent the second end 114 of the switch body 102 and prevents removal of the switch button member 106 from that second end 114. The switch button member 106 is substantially cylindrically configured and is inserted through the first end 112 of the switch body 102. The outer shoulder 128 on the switch button member 106 will engage the first internal shoulder stop 122 and that shoulder stop does not allow the switch button member to pass beyond that point and be removed through the opening at the second end 114.

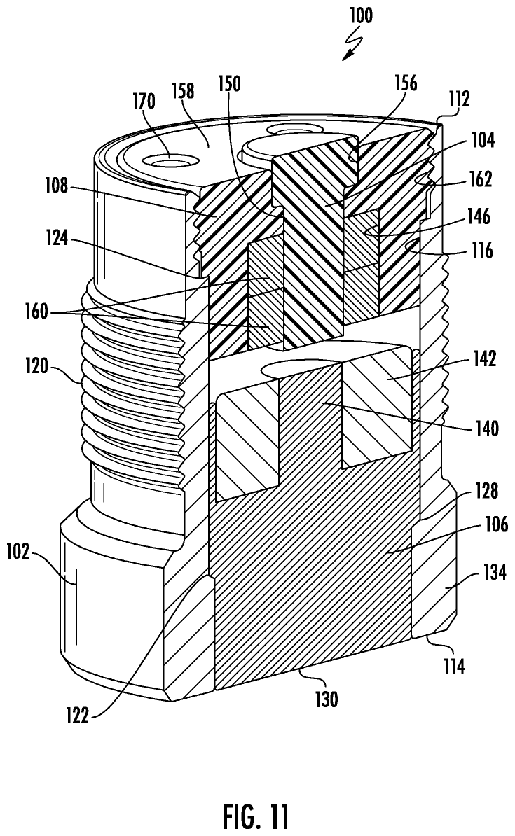

As shown in FIG. 11, when the outer shoulder 128 engages the first internal shoulder stop 122, the planar configured surface 130 formed at the end of the switch button member 106 is aligned with the end of the switch body 102 so that it forms an aesthetic and pleasing appearance such as shown in FIG. 2.

As best illustrated in FIGS. 1, 3 and 10-13, the somewhat annular configured switch body 102 includes an enlarged diameter section 134 that is similar in diameter to the outer diameter of the male tube member 36. The lower end forming the second end 114 is exposed below the lower end of the male tube member 36, such that the exposed portions of the female tube member 38 and male tube member 36, and the exposed portion of the switch button member 106 are about the same diameter and have a smooth looking surface such as shown in FIG. 1 as a mod device 30.

An annular well 134 is formed in the switch button member 106 on the section opposite the second end 114 and forms a central post 140. A first magnet 142 of a first polarity is received within that annular well. The first magnet 142 is annular configured and has an opening through which the central post 140 of the switch button member 106 is received or passes as shown in FIG. 11. The height of the first magnet 142 is substantially the same as the height of the central post 140 as illustrated so that the top surface of the first magnet and the central post are substantially coplanar to each other forming a substantially planar surface in this illustrated example. The diameter of the opening in the first magnet 142 has a tolerance that allows the first magnet to be received over the central post 140, but with little clearance so that there is no substantial movement of the first magnet within the annular well. The outer diameter of the first magnet 142 is chosen to permit a slight clearance fit within the annular well as illustrated.

The switch body 102 and switch button member 106 are formed of a conductive material, such as a metallic material, that could be as an example stainless steel, brass or copper. The switch insulator 108 is secured within the first opening at the orifice 116 in the switch body 102 at the first end 114 and the switch insulator includes a central orifice 144 and internal couterbore section 146 opposite the first end 112 of the switch body 102. A shoulder 150 is formed within the central orifice 144 of the insulator 108 adjacent the internal counterbore section 146 and juts out as a rectangular configure protrusion. The switch firing pin 104 is received within the central orifice 144. As best shown in FIGS. 7, 8 and 14, the switch firing pin 104 includes a notch 152 that is rectangular configured and slightly less in diameter than the firing pin lower section end that receives the shoulder 150 and retains the switch firing pin within the central orifice 144. The notch 152 has a length that is slightly greater than the height of the shoulder 150, such as shown in FIG. 14, so that there is some slight play allowing vertical movement of the switch firing pin 104 relative to the switch insulator 108.

In an example, this height and clearance for the switch firing pin 104 and its notch 152 relative to the shoulder 150 is about 0.010 inches to 0.030 inches, and in one non-limiting example, is about 0.014 inches. The shoulder 150 limits upward movement of the switch firing pin 104 out of the central orifice 144. The shoulder 150 of the switch insulator 108 has an internal diameter that is less than the outer diameter of the lower section of the switch firing pin 104 that extends below the shoulder, which is a few thousands of an inch greater than the internal diameter of the switch insulator shoulder 150. This difference requires the switch firing pin 104 to be pressed into the central orifice 144 so that the lower section of the switch firing pin below the notch 152 may not pass beyond the shoulder 150 within the central orifice 144 adjacent the internal counterbore section 146. The switch firing pin 104 includes a firing pin head 154 that has a greater diameter than the firing pin itself and is received within an upper counterbore section 156. The switch insulator has an upper shoulder ridge formed on the shoulder 150 and the firing pin head 154 has a lower annular ridge that engages the upper shoulder ridge. The switch insulator 108 includes an outer planar surface 158 (FIG. 11) and the firing pin head 154 that extends slightly above the outer planar surface, although it could be somewhat coplanar depending on design. It has been found that having the firing pin head 154 extending slightly above the outer planar surface 158 ensures even better control over the vaping operation and electrical actuation of the coils.

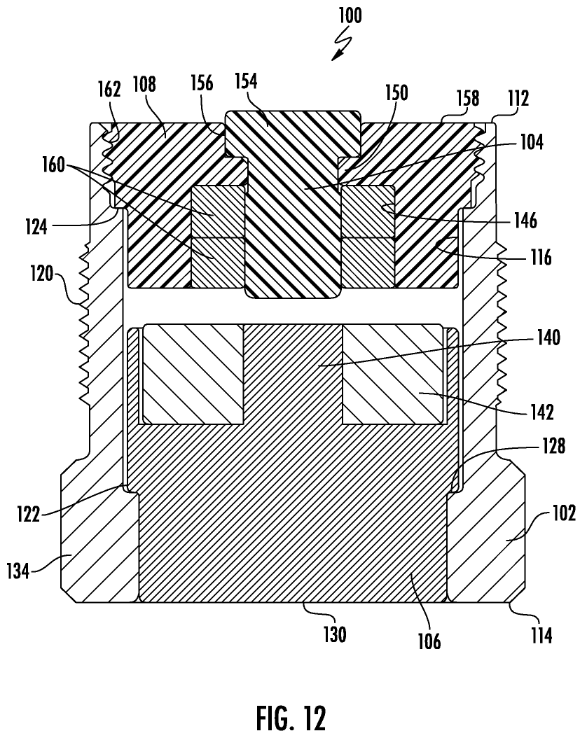

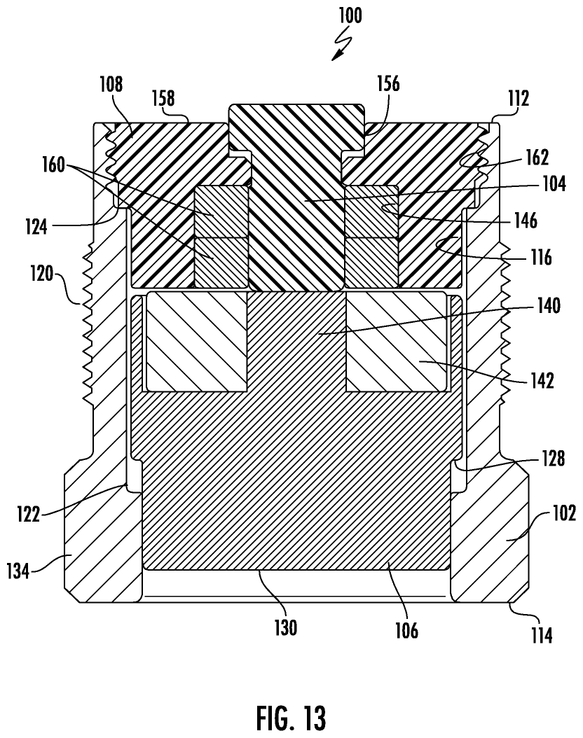

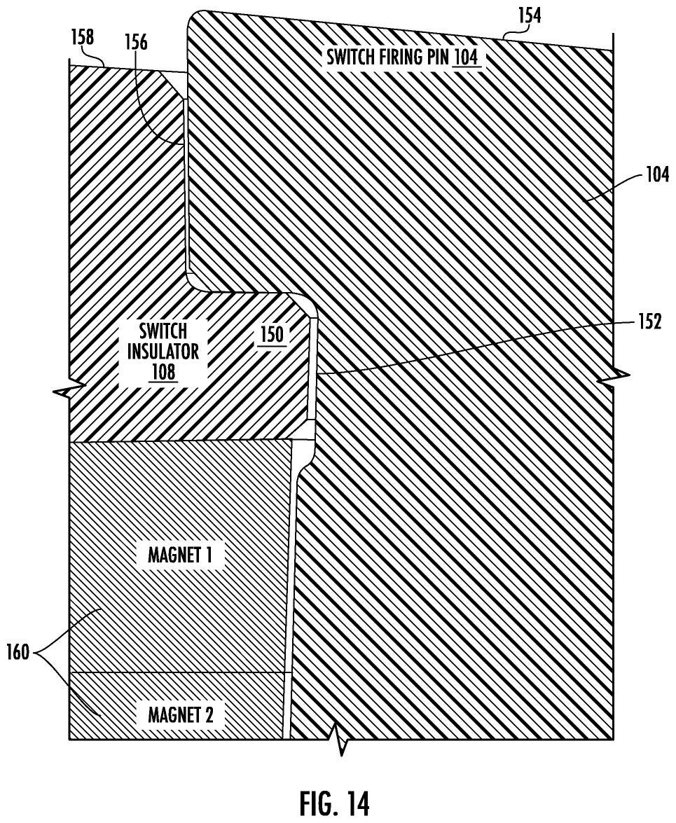

At least one second magnet 160 is received within the internal or lower counterbore section 146 and has a polarity opposite the first magnet 142. In an example, the at least one second magnet 160 is annular configured and includes a donut hole opening through which the lower section of the firing pin 104 is received, and in an example, the at least one second magnet includes two stacked magnets retained within the internal or lower counterbore section 146. The switch button member 106 and its central post 140 are thus repelled away from the switch firing pin 104 and switch insulator 108 as shown in FIGS. 11 and 12. When a user depresses the switch button member 106 as shown in FIG. 13 where the switch assembly 100 is in its closed state, contact is made between the central post 140 and switch firing pin 104 to complete the electrical circuit with the battery 56 received within the mod housing. When the switch button member 106 is depressed inward, the switch firing pin 104 engages the shoulder as a stop 150, which limits upward movement of the switch firing pin out of the central orifice 144, as shown in FIGS. 12 and 13, of the switch insulator 108, illustrating how the slight clearance of a few thousands of an inch between the notch 152 and shoulder 150 permits the firing pin head 154 to move vertically or elevate a few more thousands of an inch and ensure even better contact without exerting undue force on the battery 56 contained in the housing. Thus, if a user exerts unnecessary upward pressure on the switch button member 106 when activating the device 30 for vaping, the shoulder 150 would limit vertical movement of the firing pin head and not allow it to extend more than a few thousands of an inch. Any excessive vertical movement of the firing pin 104 could cause damage to the device. An example would be exerting undue force on the battery terminal by pressing the firing pin 104 against the battery 56 with undue upward force and exert excessive force in turn onto the RDA subassembly mounted on the mod housing.

As illustrated, the switch body 102, switch button member 106 and switch insulator 108 are all substantially cylindrically configured. The switch insulator 108 includes a threaded upper section 162 that is received within the threaded opening at the first end of the switch body 102. The outer planar surface 158 of the switch insulator as illustrated and includes tool receiving orifices 170 or other tool attachment mechanism that receive a tool that is configured to remove the switch insulator 108 from the switch body 102, such as unscrewing the switch insulator from the switch body and can be used as vent holes for battery gases.

As noted before and described in the incorporated by reference '676 patent application, a three-piece button assembly for the switch assembly 100 may be used. Different ornamental features may be applied onto the outer planar surface 130 that a user depresses on the switch button member 106. The switch insulator 108 can be formed from any insulator material such as a plastic material. In an example, the insulator material forming the switch insulator 108 may be formed from a plastic material that could include polyetherimide as an amber-to-transparent solid often used with 3D printers and referred to by the tradename Ultem and have high heat resistance, solvent resistance, flame resistance and have a high dielectric strength. Other materials could include Delrin as polyoxymethylene, also referred to as Celcon and by other tradenames. It has high strength, hardness and rigidity and is available in different colors. Acrylic could be used. Many other plastics could be used. For all components, normal manufacturing tolerances may apply.

The clearances and tolerances between the outer dimensions of the switch button member 106 and internal dimensions of the orifice 116 of the switch body allow sufficient clearance and permit the switch button member to be depressed and thus moved vertically within the orifice 116. The first and second magnets 142,160 have sufficient magnetic strength to impart a good “feel” to the user when depressing the button member, but at the same time not requiring excess force to be necessary for depressing the switch button member.

Many modifications and other embodiments of the invention will come to the mind of one skilled in the art having the benefit of the teachings presented in the foregoing descriptions and the associated drawings. Therefore, it is to be understood that the invention is not to be limited to the specific embodiments disclosed, and that the modifications and embodiments are intended to be included within the scope of the dependent claims.

That which is claimed is:

1. A personal vaporizer mod device, comprising:
a mod housing comprising a closed upper end having a medially located orifice configured to receive an atomizer pin of a rebuildable atomizer deck that is supported on the upper end, and an open lower end configured to receive a battery having a battery contact that engages the atomizer pin for supplying electric current to the rebuildable atomizer deck for atomizing a vaping liquid; and
a switch assembly received in the open lower end of the mod housing which when actuated, completes an electrical circuit from the battery into the rebuildable atomizer deck, said switch assembly comprising:
a switch body having first and second ends and an orifice defining respective first and second openings, the first end received within the lower end of the mod housing, and an internal shoulder stop within the central orifice proximal the second end;
a switch button member received within the orifice and movable therein, and including an outer shoulder that engages the internal shoulder stop and prevents removal of the switch button member from the opening at the second end, an annular well forming a central post, and a first magnet of first polarity received within the annular well; and
a switch insulator secured within the opening of the switch body and having a central orifice and an internal counterbore section, and a shoulder within the central orifice adjacent the internal counterbore section, a switch firing pin received within the central orifice, and including a notch that receives the shoulder and retains the switch firing pin within the central orifice, and at least one second magnet received within the internal counterbore section and having a polarity opposite the first magnet, wherein the switch button member and central post are repelled away from the switch firing pin, wherein upon depression of said switch button member, contact is made between the central post and switch firing pin to complete an electrical circuit to the battery received within the mod housing.

2. The personal vaporizer mod device according to claim 1 wherein said mod housing comprises a conductive female tube member and conductive male tube member forming a hybrid sleeve.

3. The personal vaporizer mod device according to claim 1 wherein the notch of the switch firing pin is configured to engage the shoulder stop and limit upward movement of the switch firing pin out of the central orifice.

4. The personal vaporizer mod device according to claim 3 wherein said shoulder stop includes an upper shoulder ridge and said switch firing pin includes a firing pin head having a lower annular ridge that engages the upper shoulder ridge.

5. The personal vaporizer mod device according to claim 1 wherein said switch insulator includes an outer planar surface, and said switch firing pin includes a firing pin head extending above said outer planar surface.

6. The personal vaporizer mod device according to claim 1 wherein said first magnet is annular configured and has an opening through which said central post of said switch button member passes.

7. The personal vaporizer mod device according to claim 1 wherein said at least one second magnet is annular configured and has an opening through which the firing pin extends.

8. The personal vaporizer mod device according to claim 7 wherein said at least one second magnet comprises two stacked magnets retained within the internal counterbore section.

9. The personal vaporizer mod device according to claim 1 wherein said switch button member includes a planar configured surface that a user depresses.

10. A switch assembly for a personal vaporizer mod device, comprising:
a switch body having first and second ends and an orifice defining respective first and second openings, wherein the first end is configured to be received within a mod housing, and an internal shoulder stop within the orifice proximal the second end;
a switch button member received within the orifice and movable therein, and including an outer shoulder that engages the internal shoulder stop and prevents removal of the switch button member from the opening at the second end, and an annular well forming a central post, and a first magnet of first polarity received within the annular well; and
a switch insulator secured within the first opening of the switch body and having a central orifice and an internal counterbore section, and a shoulder within the central orifice adjacent the internal counterbore section, a switch firing pin received within the central orifice, and including a notch that receives the shoulder and retains the switch firing pin within the central orifice, and at least one second magnet received within the internal counterbore section and having a polarity opposite the first magnet, wherein the switch button member and central post are repelled away from the switch firing pin, wherein upon depression of said switch button member, contact is made between the central post and switch firing pin to complete an electrical circuit to the battery received within the mod housing.

11. The switch assembly according to claim 10 wherein the notch of the switch firing pin is configured to engage the shoulder stop and limit upward movement of the switch firing pin out of the central orifice.

12. The switch assembly according to claim 10 wherein said shoulder stop includes an upper shoulder ridge and said switch firing pin includes a firing pin head having a lower annular ridge that engages the upper shoulder ridge.

13. The switch assembly according to claim 10 wherein said switch insulator includes an outer planar surface, and said switch firing pin includes a firing pin head extending above said outer planar surface.

14. The switch assembly according to claim 13 wherein said outer planar surface of said switch insulator is configured to receive a tool to remove the switch insulator from the switch body.

15. The switch assembly according to claim 10 wherein the switch body, switch button member and switch firing pin are formed of a conductive material.

16. The switch assembly according to claim 10 wherein said first magnet is annular configured and has an opening through which said central post of said switch button member passes.

17. The switch assembly according to claim 10 wherein said at least one second magnet is annular configured and has an opening through which the firing pin extends.

18. The switch assembly according to claim 10 wherein said at least one second magnet comprises two stacked magnets retained within the internal counterbore section.

19. The switch assembly according to claim 10 wherein the switch body, switch button member and switch insulator are substantially cylindrically configured.

20. The switch assembly according to claim 10 wherein said switch button member includes a planar configured surface that a user depresses.
